(19) United States
(12) Patent Application Publication
(10) Pub. No.: US 2003/0175687 A1
(43) Pub. Date: Sep. 18, 2003

(54) Methods for the detection and identification of microorganisms

(76) Inventor: Janet Mary Tippet, Ames, IA (US)

Correspondence Address: W. Robinson H. Clark, DORSEY & WHITNEY, LLP, # 300 South, 1001 Pennsylvania Avenue, N.W., Washington, DC, 20004, US

(21) Appl. No.: 10/094,803

(22) Filed: Mar. 12, 2002

(51) Int. Cl. C12Q 1/70; G06F 19/00; G01N 33/48; G01N 33/50; C12Q 1/04

(52) U.S. Cl. 435/5; 435/34; 702/19

(57) ABSTRACT

This invention relates to methods for rapidly evaluating and identifying a microorganism of interest. A preferred method comprises (1) providing a plurality of said microorganism to a flow cytometer, wherein the plurality of said microorganism are labeled with a fluorescent marker; (2) obtaining from the flow cytometer fluorescence and scatter signals to generate a profile of said microorganism; and (3) comparing the profile thus generated to a signature profile of a reference microorganism. This invention is also directed to a microorganism evaluation system which comprises (1) a means for gathering fluorescence and scattering signals of a microorganism of interest; and (2) a means for comparing said fluorescence and scatter signals to a signature profile of a reference microorganism.

### TECHNICAL FIELD

This invention relates to methods for rapidly detecting, evaluating and identifying microorganisms. The method employs flow cytometry.

### BACKGROUND

Traditional methods for microbial detection and identification involve forming a culture in growth media and using biochemical or immunological tests to identify the microorganism of interest. These methods are time-consuming. In addition, traditional methods frequently use tags that are specific to the microorganism of interest. For instance, several methods use antibody-based testing to identify the microorganism by its antigenic properties, or use nucleic acid probes for specific DNA or RNA sequences of the microorganism.

One method uses a general and non-specific test for hygiene monitoring. This method involves the use of adenosine triphosphate (ATP) bioluminescence. However, this method provides only an indication of overall cleanliness of a test area, and is insufficient for determining the identity of the microorganisms that are associated with the test area.

Flow cytometry is used for evaluating microorganisms. For instance, Optoflow (Optoflow, Norway) provides a flow cytometry system for counting cells. See www.optoflow.com. However, this system does not attempt to identify microorganisms. Another flow cytometry system, Partec CyFlow Cytometer, is provided by Partec GmbH in Denmark. See www.partec.de/applications.

Accordingly, there is a need to provide a system capable of identifying a microorganism of interest without using specific fluorescent labels. In addition, there is a need to provide a system capable of identifying a microorganism of interest rapidly.

### SUMMARY OF THE INVENTION

It is an object of this invention to provide a system that is capable of evaluating a microorganism rapidly, and preferably, without using specific labeling markers. In accordance with one aspect of this invention, the method comprises: (1) providing a plurality of said microorganism to a flow cytometer, the plurality of said microorganism being labeled with a fluorescent marker; (2) obtaining from the flow cytometer fluorescence and scatter signals to generate a profile of said microorganism; and (3) comparing the generated profile to a signature profile of a reference microorganism.

In one embodiment, the generated profile of said microorganism comprises a bitmap of said microorganism. In addition, the signature profile of said reference microorganism comprises a bitmap of said reference microorganism. The microorganism is identified by comparing the generated profile to the signature profile. This can be done using a pattern recognition program.

In another embodiment, the generated profile of said microorganism comprises a ratio of counts in a predetermined box in a bitmap of said microorganism over total counts in the bitmap of said microorganism. In addition, the signature profile of said reference microorganism comprises a range which encompasses a ratio of counts in the predetermined box in a bitmap of said reference microorganism over total counts in the bitmap of said reference microorganism. Once again, the microorganism is identified by comparing the generated profile to the signature profile.

In yet another embodiment, the generated profile further comprises a mean, a standard deviation and a coefficient of variance of the fluorescence intensity in the predetermined box in said microorganism's bitmap, and a mean, a standard deviation and a coefficient of variance of the scatter intensity in the predetermined box in said microorganism's bitmap. The signature profile may comprise a first, second and third range which encompass a mean, a standard deviation and a coefficient of variance, respectively, of the fluorescence intensity in the predetermined box in said reference microorganism's bitmap, and a fourth, fifth and sixth range which encompass a mean, a standard deviation and a coefficient of variance, respectively, of the scatter intensity in the predetermined box in said reference microorganism's bitmap.

In a preferred embodiment, the generated profile is compared to the signature profile using a programmable processor.

The selected marker may be specific. However, it is preferable to use a marker that is not specific to the microorganism being identified and the reference microorganism.

The microorganisms may be capable of causing food poisoning or food spoilage. In one embodiment, the reference microorganism is selected from Listeria innocua, Enterococcus faecium, Pseudomonas aeruginosa and Candida albicans.

In one embodiment, the method comprises obtaining a plurality of said microorganism in a selection culture medium. The method also comprises comparing the generated profile of said microorganism to a plurality of signature profiles of reference microorganisms.

In accordance with yet another aspect of this invention, there is provided with a system for evaluating a microorganism. The system comprises: (1) a means for gathering fluorescence and scattering signals of said microorganism; and (2) a means for comparing said fluorescence and scatter signals to a signature profile of a reference microorganism. The means for gathering may comprise a flow cytometer, and the means for comparing may comprise a programmable processor.

Other features, objects, and advantages of the present invention are apparent in the detailed description that follows. It should be understood, however, that the detailed description, while indicating preferred embodiments of the invention, are given by way of illustration only, not limitation. Various changes and modifications within the spirit and scope of the invention will become apparent to those skilled in the art from the detailed description.

### BRIEF DESCRIPTION OF THE DRAWINGS

The file of this patent or patent application contains at least one drawing executed in color. Copies of this patent or patent application publication with color drawing(s) will be provided by the Patent and Trademark Office upon request and payment of the necessary fee. The drawings are provided for illustration, not limitation.

FIG. 1 illustrates a bitmap of Listeria innocua.

FIG. 2 shows a side scatter plot of the bitmap of FIG. 1.

FIG. 3 depicts a fluorescence intensity plot of the bitmap of FIG. 1.

FIG. 4 is a flow chart depicting a process for comparing a signature profile of Listeria innocua to a profile of a testing microorganism.

FIG. 5 illustrates a bitmap of Staphylococcus aureus.

FIG. 6 shows a bitmap of Enterococcus faecalis.

FIG. 7 is a bitmap of Enterococcus faecium.

FIG. 8A shows a bitmap of Sample S1.

FIG. 8B depicts a bitmap of Sample S2.

FIG. 8C is a bitmap of Sample S3.

FIG. 8D is a bitmap of Sample S4.

### DETAILED DESCRIPTION

This invention relates to methods for fast detection and identification of microorganisms. In the preferred method, a microorganism of interest is enriched or multiplied, and labeled with a fluorescent tag. The fluorescent tag may be specific, but is preferably non-specific, to the microorganism of interest. A sample of the fluorescence labeled microorganism is then introduced to a flow cytometer. The fluorescence and scatter signals are then gathered from the flow cytometer and used to generate a signature profile of the microorganism. The signature profiles are then compared to unidentified microorganisms of interest for quick identification of the microorganism.

As used herein, the signature profile may be a two-dimensional bitmap having one axis representing the fluorescence intensity and the other axis representing the scatter intensity. The signature profile may also comprise a series of elements corresponding to, for example, the features of the distribution of the fluorescence or scatter intensity. These features may include the mean, standard deviation and coefficient of variance of the fluorescence or scatter intensity. Other features of the fluorescence and scatter signals may also be used to generate the signature profile of the microorganism of interest, provided that the signature profile thus generated is useful to distinguish the microorganism of interest from other microorganisms.

As used herein, a microorganism having a signature profile may be referred to as a reference microorganism. A bitmap refers to not only the physical bitmap that is printed out from the flow cytometer, but also any data form which is stored in a storage medium and which corresponds to or encodes the physical bitmap. Such a data form may be either derived from the physical bitmap, or capable of generating the physical bitmap. Such a data form may be stored or organized in any forms, as appreciated by one of skill in the art. Similarly, a box within a bitmap can be in any data form. Likewise, an element in a signature profile, or a value in a profile, refers to not only a range or a number, but also any data form which is stored in a storage medium and which corresponds to or encodes said range or number.

Different microorganisms may have different signature profiles. Different signature profiles can be organized into a profile library and stored in a storage medium, as appreciated by one of ordinary skill in the art. A profile of a testing microorganism may be similarly obtained, and compared to the profile library in order to evaluate the identity of the testing microorganism. The comparison may preferably be carried out using a computer or a programmable processor. The comparison may also be performed using eyes.

The comparison provides valuable information for determining the identity of a testing microorganism. For instance, different species of Listeria tend to have a similar signature profile. Thus, where the profile of the testing microorganism matches such a signature profile, it is likely that the testing microorganism belongs to Listeria. For another instance, different species of Enterococcus tend to have different signature profiles. Consequently, where the profile of the testing microorganism matches the signature profile of a particular species of Enterococcus, it is likely that the testing microorganism is identical to the particular Enterococcus species. The comparison of a profile of an unknown microorganism to that of a known microorganism allows fast and easy evaluation of the identity of the unknown microorganism.

The methods of this invention may also be used to detect the presence or absence of a microorganism of interest in a biological sample. For instance, the microorganisms in the biological sample may be isolated and multiplied using conventional means. The profiles of these microorganisms are obtained and compared to the signature profile of the microorganism of interest so as to determine if the biological sample contains the microorganism of interest.

Any commercial flow cytometer may be used in this invention. In one embodiment, the flow cytometer model RBD2100, manufactured by Advanced Analytical Technologies Inc. (2901 S. Loop Drive, Ames, Iowa 50010) is used to gather and analyze the fluorescence and scatter signals. As used herein, a flow cytometer may comprise a computer or a programmable processor, whether or not physically integrated, to analyze fluorescence and scatter signals and to obtain desired parameters, such as mean, SD or CV of the fluorescence or scatter intensity.

Any fluorescent tag may be used to label a microorganism of interest. For instance, non-specific tags can be employed. Non-specific tags include, but are not limited to, Draq5 (manufactured by Biostatus Limited, U.K.) and SYTO 62 (manufactured by Molecular Probes, Inc., Eugene, Oreg.).

Microorganisms may be prepared using conventional methods, such as those described in Difco Manual (11^{th} ed., Becton Dickinson and Company, Sparks Md.), which is incorporated herein by reference. Microorganisms that can be detected by this invention include, but are not limited to, bacteria and yeast, such as Staphylococcus, Listeria, Enterococcus and Candida. The identity of the microorganisms may be confirmed using biochemical or immunological tests, such as those described in Bergey's Manual of Determinate Bacteriology (9^{th} ed., 1994, Williams and Wilkins), which is incorporated herein by reference.

Preferably, microorganisms are labeled with fluorescent tags when they grow to log phase. In one embodiment, selective media are used to select the growth of the microorganism of interest while inhibiting the growth of other microorganisms. For instance, Listeria species may be enriched or selected using the methods described in FDA Bacteriological Analytical Manual (1995) or USDA/FSIS Microbiology Guidebook (3^{rd} ed. 1998), both of which are incorporated herein by reference.

The fluorescence-labeled microorganisms are injected into a flow cytometer. The concentration of the injected microorganisms may be in the range of about 10⁴ microorganisms per ml. The concentration may be below 10⁴ microorganisms per ml. In one embodiment, the concentration of the injected microorganisms is between about 10⁵ and about 10⁶ microorganisms per ml. Fluorescence and side scatter signals may be captured and analyzed using software associated with the flow cytometer. Preferably, the mean, standard deviation (SD) and coefficient of variance (CV) of the fluorescence and side scatter signals are obtained. These values may be used to construct the elements of a signal profile of the microorganisms. The confidence levels for side scatter and fluorescence measurements may be set at no less than 0.90, such as 0.95 and 0.99.

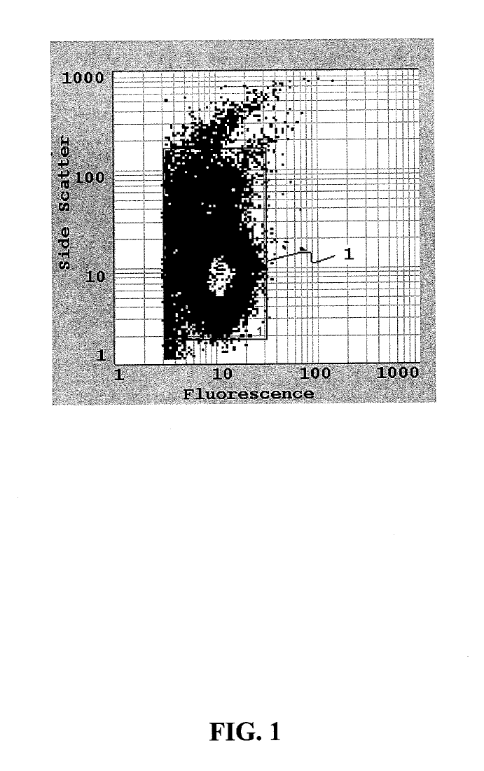

FIG. 1 shows a bitmap of Listeria innocua which is labeled with Draq5 and detected using the RBD2100 model flow cytometer. The x-axis of the bitmap represents fluorescence intensity, and the y-axis denotes side scatter intensity. The counts of the microorganisms at different fluorescence and scatter intensities are shown on a color scale, from low to high, represented by blue, green, yellow and red. The box in the bitmap (Box 1) captures about 97% of fluorescence and scatter signals.

As used in this invention, Box 1 may be drawn to capture any predetermined percentage of fluorescence or scatter signals. For instance, Box 1 may be designed to encompass about 90%, 95%, 96%, 97% or 99% of fluorescence or scatter signals. Preferably, Box 1 is drawn to exclude background signals. More preferably, Box 1 encompasses the majority of the fluorescence and scatter signals. Means, standard deviations, coefficients of variance, or other features of the fluorescence and scatter intensity may be measured or determined based on the counts within Box 1. Two or more boxes may be drawn in the bitmap, and the data obtained therefrom may be used to construct signature profiles.

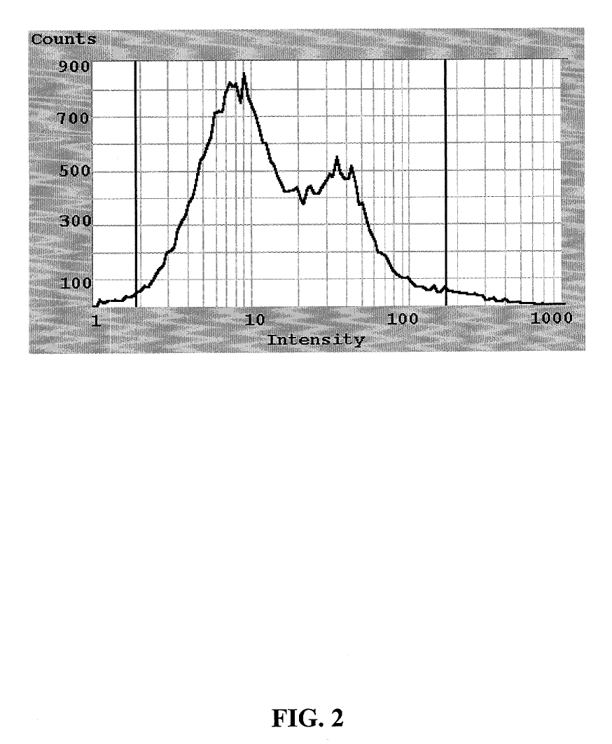

FIG. 2 shows a side scatter plot of the bitmap of FIG. 1. The x-axis represents scatter intensity, and the y-axis shows counts at each scatter intensity. The two vertical lines in the side scatter plot correspond to the two horizontal sides of Box 1. The mean of the scatter intensity between the two vertical lines, i.e. within Box 1, may be calculated. The confidence level may be set at a predetermined value, such as 95%. The standard deviation (SD) and coefficient of variance (CV) of the scatter intensity between the two vertical lines may also be determined. For the scatter plot of FIG. 2, the mean, SD and CV thus determined are about 13, 14 and 193, respectively.

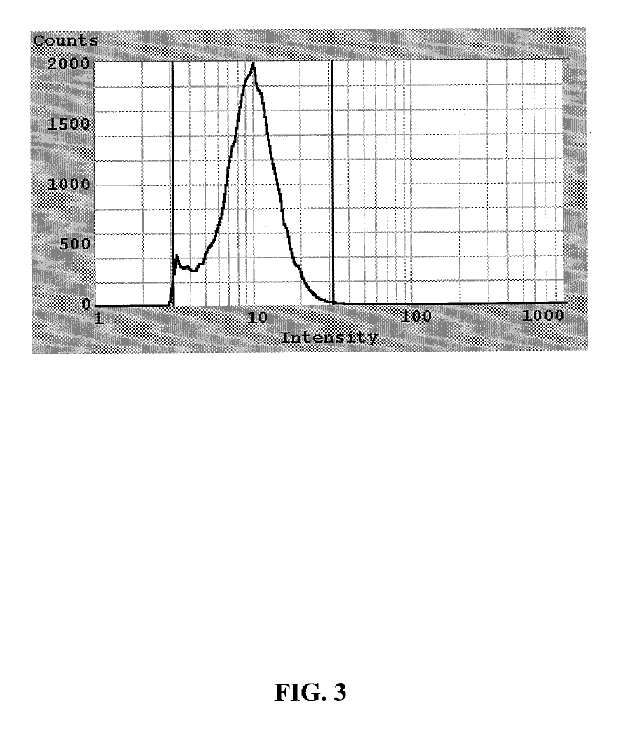

FIG. 3 is a fluorescence intensity plot of the bitmap of FIG. 1. The x-axis represents fluorescence intensity, and the y-axis demonstrates counts at each fluorescence intensity. The two vertical lines in the fluorescence intensity plot correspond to the vertical sides of Box 1. The mean of the intensity of the fluorescence signals between the two vertical lines may be calculated. The confidence level can be set at a predetermined value, such as 95%. The standard deviation (SD) and coefficient of variance (CV) of the intensity of the fluorescence signals between the two vertical lines may also be determined. For the fluorescence intensity plot of FIG. 3, the mean, SD and CV thus determined are about 9, 4 and 48, respectively.

The data obtained from FIGS. 1-3 are summarized as follows:

Sample: Listeria innocua

Sample Time (second): 4.8×10²

Sample Loop Size: 250 μL

Fluorescence High Voltage (DPot): 88

Scatter High Voltage (DPot): 115

Fluorescence Threshold (DPot): 10

Fluorescence Lower Limit: 3

Fluorescence Upper Limit: 32

Side Scatter Lower Limit: 0.86

Side Scatter Upper Limit: 168.19

Total Counts: 3.2×10⁴

Box 1 Counts (97% total count): 3.1×10⁴

Side Scatter Confidence Level: 0.95

Side Scatter Coefficient of Variance: 193

Side Scatter Mean: 13

Side Scatter Standard Deviation: 14

Side Scatter Confidence Interval: no less than 12.77 but no greater than 13.23

Fluorescence Confidence Level: 0.95

Fluorescence Coefficient of Variance: 48

Fluorescence Mean: 9

Fluorescence Standard Deviation: 4

Fluorescence Confidence Interval: no less than 8.96 but no greater than 9.04

The means, standard deviations (SD) and coefficients of variance (CV) of the fluorescence and scatter signals within Box 1 may be used to generate a signature profile of Listeria innocua. The corresponding values obtained from an unknown microorganism using the flow cytometer may be compared to the signature profile of Listeria innocua to determine if the unknown microorganism is Listeria innocua.

The signature profile of Listeria innocua may also comprise the ratio of the counts in Box 1 over the total counts in the bitmap of FIG. 1. A box having the same position and size as Box 1 may be drawn in the bitmap of the unknown microorganism. The ratio of the counts in the box thus drawn over the total counts in the bitmap may be determined and compared to the corresponding ratio derived from Box 1 in FIG. 1.

Therefore, at least seven elements, including the means, SDs and CVs of the fluorescence and scatter intensity within a predetermined box, and the ratio of counts in the predetermined box over the total counts in the bitmap, may be used to construct a signature profile of a microorganism of interest. As appreciated by one of ordinary skill in the art, these elements can be determined using a computer or a programmable processor, whether or not it is associated with the flow cytometer.

Preferably, each element in the signature profile is represented by a range, instead of a definite number. For instance, the range corresponding to a given value, such as the mean of the intensity of the fluorescence signals, may be between 60% and 140% of the given value. Preferably, it is between 70% and 130% of the value. More preferably, it is between 80% and 120% of the value. Highly preferably, it is between 90% and 110% of the value. In one embodiment, it is between 95% and 105% of the value.

Moreover, the ranges in a signature profile may be different from one another. For instance, the range corresponding to the mean of the intensity of the fluorescence signals may be between 85% and 115% of the mean value, while the range corresponding to the SD of the fluorescence intensity may be between 75% and 125% of the SD value. Furthermore, the range of a given value may be symmetrical or asymmetrical in relation to the value. For instance, the range corresponding to the mean of the scatter intensity may be between 80% and 140% of the mean value.

In one embodiment, the range of a given value is determined by first measuring the value several times to obtain different results and then selecting a range within which all of these results are included. For instance, the mean of the scatter intensity within Box 1 may be measured several times using the same microorganism. Each time, the mean may be different. A range is then chosen to encompass all of the different means. As appreciated by one of ordinary skill in the art, the accuracy of identification may decrease as the ranges used in the signature profile increase.

A signature profile may comprise less than, or more than, seven elements. As the number of the elements decreases, the accuracy of identification may decrease.

A similarly obtained profile of an unknown microorganism may be compared to each element in the signature profile of a microorganism of interest. For instance, a box which has the same size and position as Box 1 may be created in the bitmap of the unknown microorganism. Fluorescence and scatter intensity within the box is analyzed to generate means, SDs, CVs, or other parameters that may reflect the features of the distribution of the fluorescence and scatter intensity within the box. These features or values are organized to form a profile of the unknown microorganism. If all of the components in the profile of the unknown microorganism match all of the corresponding elements in the signature profile of the microorganism of interest (for instance, all of the components in the profile are located within all of the corresponding ranges in the signature profile), then it is likely that the unknown microorganism is identical or related to the microorganism of interest.

In one embodiment, the unknown microorganism may be considered to match the microorganism of interest if the profile of the unknown microorganism matches a majority of the elements of the signature profile, such as six out of seven elements. One of ordinary skill in the art would understand that the number of the elements, satisfaction of which is required before the unknown microorganism is considered to match, may be determined depending on the need of accuracy.

In another embodiment, the signature profile comprises elements obtained from at least two boxes created in the bitmap of the microorganism of interest. The profile of the unknown microorganism, therefore, is derived from at least two corresponding boxes in the unknown microorganism's bitmap.

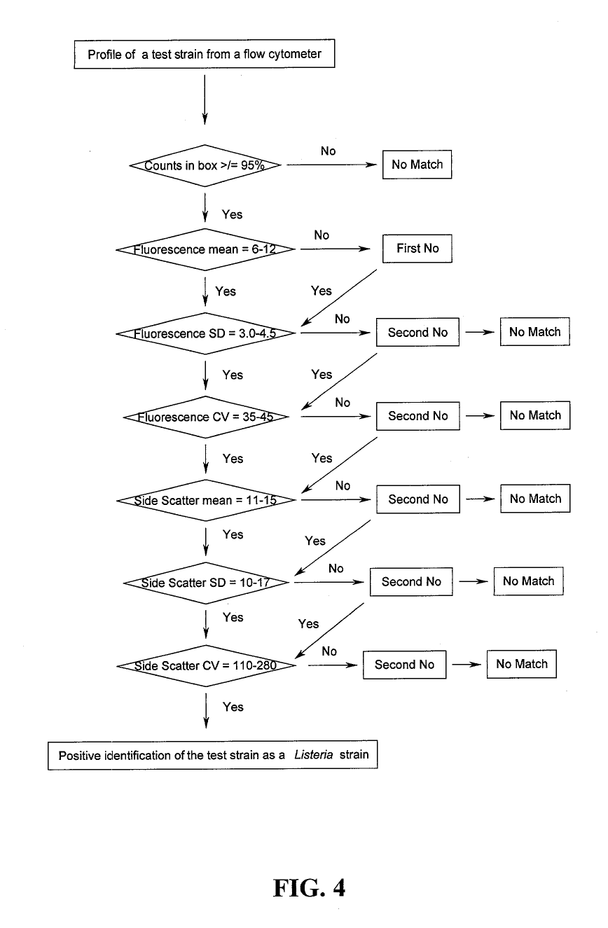

In a preferred embodiment, the comparison between the signature profile of the microorganism of interest and the profile of the unknown microorganism is carried out using a computer or a programmable processor. FIG. 4 depicts a flow chart showing a process of comparing a signature profile of Listeria innocua to the profile of a testing microorganism. The signature profile used in FIG. 4 comprises the ranges corresponding to the ratio of counts in Box 1 of FIG. 1 over total counts in the bitmap of FIG. 1 (no less than 95%), the mean of the fluorescence intensity in Box 1 (6-12), the SD of the fluorescence intensity in Box 1 (3.0-4.5), the CV of the fluorescence intensity in Box 1 (35-48), the mean of the scatter intensity in Box 1 (11-15), the SD of the scatter intensity in Box 1 (10-17), and the CV of the scatter intensity in Box 1 (110-280). The profile of the testing microorganism is similarly determined based on a predetermined box in the bitmap of the testing microorganism, wherein the predetermined box has the same position and size as Box 1.

The steps in FIG. 4 are designed to determine whether there is a match between the testing microorganism and Listeria innocua. For the test strain to be considered for further comparison, it must satisfy the requirement that no less than 95% of counts is captured within the predetermined box. If this criterion is not met, there is no match and no further comparisons to the remaining criteria are made. If the criterion of no less than 95% counts being within the box is met, then the comparison cascade is continued. For the example in FIG. 4, there are six further criteria. If any five of these six criteria are met, a match between the testing microorganism and Listeria innocua is considered to be made. Although FIG. 4 uses a signature profile of Listeria innocua, this process, or a modification thereof as appreciated by one of ordinary skill in the art, is generally applicable to other microorganisms.

Signature profiles of microorganisms other than Listeria innocua can be similarly obtained. For instance, a signature profile of Enterococcus faecium is obtained from its bitmap and is shown in TABLE 1, wherein the position and size of the predetermined box is identical to Box 1 in FIG. 1.

TABLE 1
Enterococcus faecium Signature Profile

| Components | Range |
|---|---|
| ratio of counts in a predetermined | no less than 96% |
| box over total counts in the bitmap | |
| mean of the fluorescence intensity | 12-15 |
| in the predetermined box | |
| SD of the fluorescence intensity in | 4.5-5.0 |
| the predetermined box | |
| CV of the fluorescence intensity in | 35-45 |
| the predetermined box | |
| mean of the scatter intensity in the | 15-18 |
| predetermined box | |
| SD of the scatter intensity in the | 8.5-9.5 |
| predetermined box | |
| CV of the scatter intensity in the | 75-85 |
| predetermined box | |

A signature profile of Pseudomonas aeruginosa is shown in TABLE 2, wherein the position and size of the predetermined box is identical to Box 1 in FIG. 1.

TABLE 2
Pseudomonas aeruginosa Signature Profile

| Components | Range |
|---|---|
| ratio of counts in a predetermined | between 90% and |
| box over total counts in the bitmap | 95% |
| mean of the fluorescence intensity | 14-18 |
| in the predetermined box | |
| SD of the fluorescence intensity in | 6-8 |
| the predetermined box | |
| CV of the fluorescence intensity in | 35-55 |
| the predetermined box | |
| mean of the scatter intensity in the | 10-15 |
| predetermined box | |
| SD of the scatter intensity in the | 8.5-11 |
| predetermined box | |
| CV of the scatter intensity in the | 80-125 |
| predetermined box | |

TABLE 3 demonstrates a signature profile of yeast Candida albicans, wherein the position and size of the predetermined box is identical to Box 1 in FIG. 1.

TABLE 3
Candida albicans Signature Profile

| Components | Range |
|---|---|
| ratio of counts in a predetermined | no greater than 15% |
| box over total counts in the bitmap | |
| mean of the fluorescence intensity | 4.5-6.5 |
| in the predetermined box | |
| SD of the fluorescence intensity in | 3-4 |
| the predetermined box | |
| CV of the fluorescence intensity in | 55-70 |
| the predetermined box | |
| mean of the scatter intensity in the | 20-40 |
| predetermined box | |
| SD of the scatter intensity in the | 160-280 |
| predetermined box | |
| CV of the scatter intensity in the | 160-280 |
| predetermined box | |

This invention demonstrates that different species of Listeria tend to have similar bitmaps. Therefore, a signature profile of Listeria innocua may be used to detect whether an unknown microorganism belongs to Listeria. The bitmaps of different species of Enterococcus tend to be distinguishable from each other. Consequently, the signature profile of a given species of Enterococcus may be used to specifically determine if an unknown microorganism is identical to the given species.

Signature profiles of different microorganisms may be used to construct a signature library. The library can be stored in a storage medium, as appreciated by one of ordinary skill in the art. A similarly obtained profile of an unknown microorganism may be compared to the signature library to determine if the unknown microorganism is identical or related to one of the microorganisms of the library.

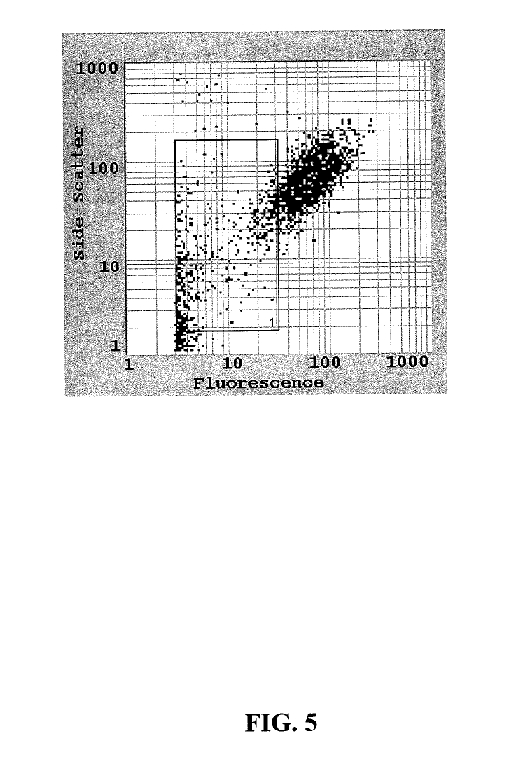

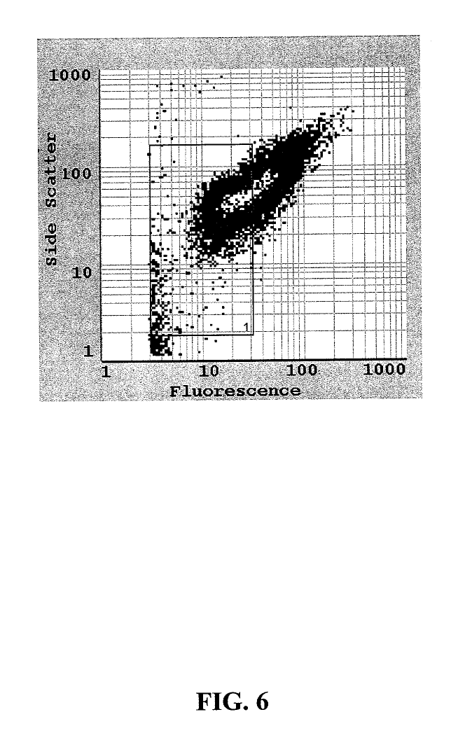

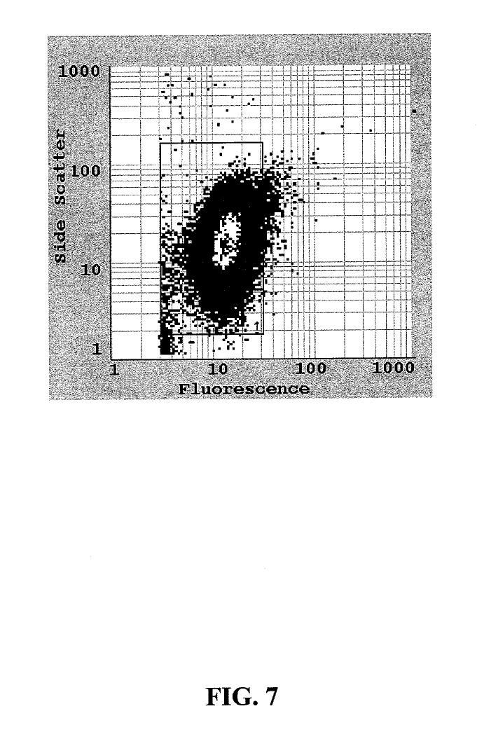

In one embodiment, the signature profile of a microorganism is its bitmap. FIGS. 5, 6 and 7 show the bitmaps of Staphylococcus aureus, Enterococcus faecalis, and Enterococcus faecium, respectively. These bitmaps are distinguishable from the bitmap of Listeria innocua as shown in FIG. 1.

The bitmaps of different microorganisms may be compared using pattern recognition or multivariate analysis. Multivariate analysis, often referred to as Chemometrics, is commonly used for two types of analysis: quantification and identification. Quantification deals with predicting the concentration of a constituent in a mixture through residual reduction techniques such as classical least squares (CLS) and partial least squares (PLS). Identification analysis deals with predicting the identity of an unknown species through pattern recognition algorithms. Pattern recognition is accomplished by accumulating a library of the instrument responses for a pure material, a mixture of materials and/or materials that have a contamination that could be present in a sample. The library can be reduced by employing algorithm such as principle component analysis (PCA). The reduction of the library, or training data set, is accomplished by describing each set of library members in N spatial degrees. These N degrees of freedom may be the flow cytometry recorded spectral properties such as: the means of fluorescence and scatter signals and the fluorescence and scatter distribution profiles. The reduced library is stored in a database and may contain examples of pure materials, mixture of materials, and contaminated materials. The reduced library is used as a comparator for unknown sample data. If the match parameters between the unknown and its nearest neighbor in the reduced library are met, the sample component is identified. The software used for pattern recognition is commercially available and is commonly employed in vibration spectrometry techniques. For references about multivariate and pattern recognition analysis, see H. Martens and T. Naes, Multivariate Calibration, John Wiley and Sons, New York, 1989; K. R. Beebe, R. J. Pell, M. B. Seasholtz, Chemometrics: A Practical Guide, John Wiley and Sons, New York, 1998; Near Infra-red Spectroscopy: Bridging the Gap between Data Analysis and NIR Applications, K. I. Hildrum, T. Isaksson, T. Naes and T. Tandberg, editors, Ellis Horwood Limideed, London, 1992; and Handbook of Near-Infrared Analysis, D. A. Burns and E. W. Ciurczak, editors, Marcel Dekker, Inc, New York, 1992, all of which are incorporated herein by reference. An example of multivariate analysis being used for microbial identification is described in Identification of Enterococcus, Streptococcus, and Staphylococcus by Multivariate Analysis of Proton Magnetic Resonance Spectroscopic Data from Plate Cultures, Journal of Clinical Microbiology 39(8): 2916-2923 (2000), which is incorporated herein by reference.

The method of this invention may be used to detect the presence or absence, or identification, of microorganisms which may be food, environmental, pharmaceutical, or clinical related. These microorganisms include, but are not limited to, bacteria, parasites, yeast and mold. For instance, they include species of Clostridia, Bacillus, Salmonella, Lactobacilli, Shigella, Campylobacter, Listeria, Vibrio, Giardia, Generic coliforms, Enterococci, Pseudomonads, Neisseria, Moraxella, Candida, Streptococci, and Staphylococci. These microorganisms, or other microorganisms of interest, may be found at various places, such as in food, beverage, soil or air, or in pharmaceutical products and clinical samples, or at bioterrorism targets. In one embodiment, the microorganism of interest is capable of causing food poisoning or food spoilage. In another embodiment, the microorganism of interest is capable of causing a human, animal or plant disease. The microorganisms may be a pharmaceutical product or a pharmaceutical contaminant. The microorganism may be obtained by culturing a sample taken from an environment of interest.

It should be understood that the above-described embodiments and the following examples are given by way of illustration, not limitation. Various changes and modifications within the spirit and scope of the present invention will become apparent to those skilled in the art from the present description.

### EXAMPLE 1

### Detecting the Presence or Absence of Listeria innocua on a Spiked Metal Surface

Listeria contamination is a major concern to the food industry. The presence of any Listeria species at critical control points (Hazard Analysis and Critical Control Point, HACCP, FDA/CFSAN) in the processing environment is an indication that the pathogen L. monocytogenes could be present, and would not have been destroyed by the processing conducted to that point. This Example investigated the recovery of Listeria innocua from a stainless steel tray that had been spiked with the organism in order to simulate sampling of an area in a food processing plant. The detection and identification was made by flow cytometry and conducted according to the process of FIG. 4, and confirmed by classical microbiology.

Bacto Letheen Broth and Bacto UVM Modified Listeria Enrichment Broth were made according to manufacturer's instructions (Difco, Becton Dickinson, Sparks, Md. 21152). Swabs used were Alpha™Swab, Texwipe 761 (Baxter Supplies). Flow cytometer was the RBD2100 model of Advanced Analytical Technologies Inc., Ames, Iowa 50010. Fluorescent dye used was Draq5™ (Biostatus Limited, U.K.). Cetyltrimethylammonium bromide was added to assist dye uptake.

A stainless steel tray (25×38 cm; 237.5 cm²) was spiked with 6.6 CFU/cm² of Listeria innocua. The tray was allowed to dry and left at ambient temperature for 3 days. For sampling, the tray was divided into 4 equal sections which were designated S1, S2, S3 and S4, respectively. Four swabs, similarly labeled S1, S2, S3 and S4, respectively, were moistened with Letheen Broth prior to swabbing their corresponding sections of the tray. The swabs were placed into individual 1 ml aliquots of Letheen Broth for 1 hour, and then were transferred to 5 ml of UVM Broth for incubation overnight. Serial dilutions of the cultures were made in filtered (0.2 micron filter) 10 mM phosphate buffer (PB), pH 7.3, to an approximate concentration of 10⁵-10⁶ organisms per ml. The diluted cultures were stained for 5 minutes with 200 μM Draq5, in the presence of 5 μM CTAB, at 37° C. in the dark. Samples were analyzed by flow cytometry using the RBD2100 model. Profiles of fluorescence and scattering signals were recorded and analyzed.

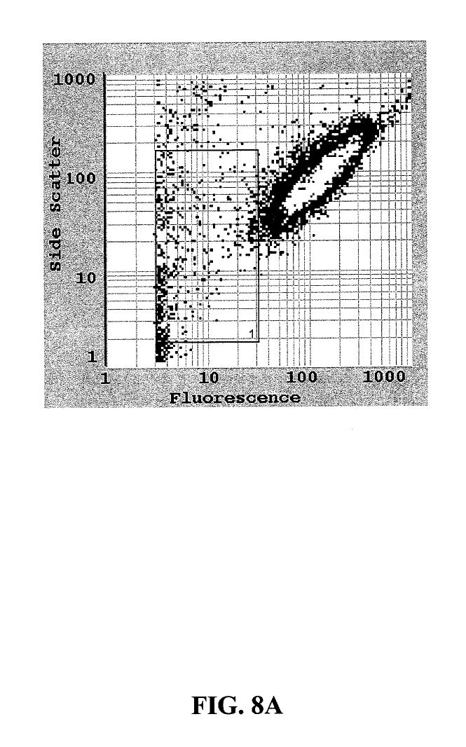

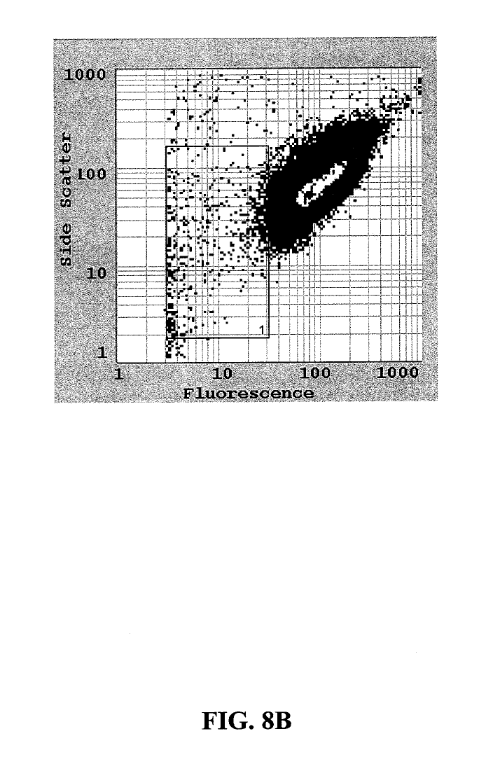

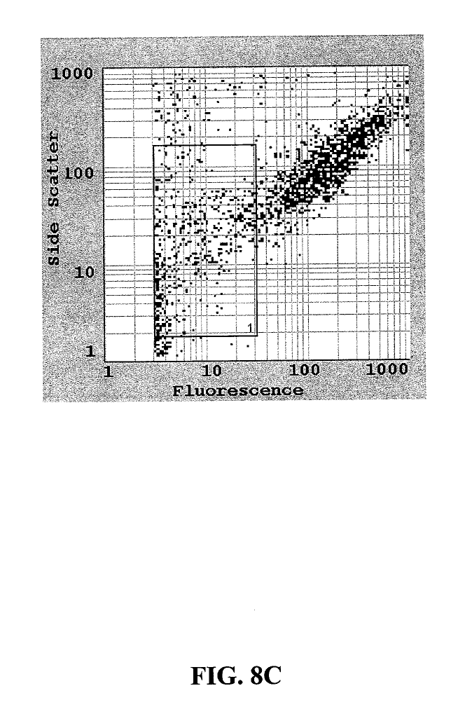

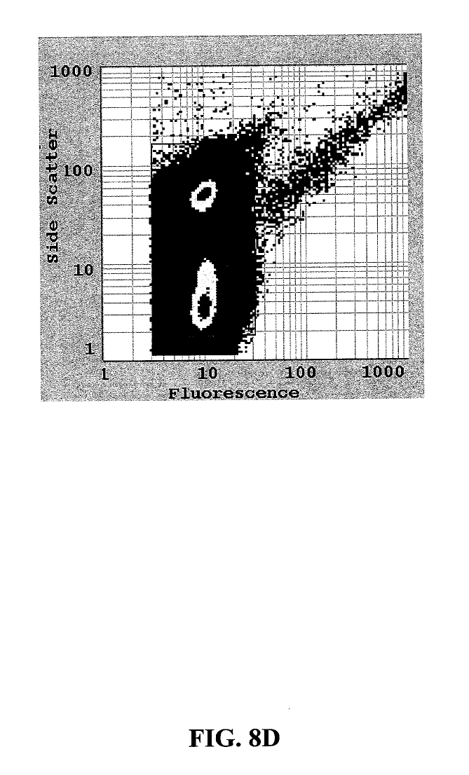

Initial bitmap profiles showed that S4 was positive for Listeria. The bitmap profiles of S1, S2 and S3 were distinctly different from Listeria. FIGS. 8A, 8B, 8C and 8D show the bitmaps of S1, S2, S3 and S4 samples, respectively. The box in each bitmap has the same position and size as Box 1 in FIG. 1.

TABLE 4 demonstrates the ratio of counts in the box over total counts in the bitmap and the means, SDs and CVs of the fluorescence and scatter intensity within the box for each of S1, S2, S3 and S4. Comparing to the signature profile of Listeria innocua used in FIG. 4, only S4 matched Listeria innocua.

TABLE 4
Profiles of Samples S1, S2, S3 and S4

| | Mean of | SD of | CV of | Mean of | SD of | CV of | Count |
|---|---|---|---|---|---|---|---|
| | Fluorescence | Fluorescence | Fluorescence | Scatter | Scatter | Scatter | Percentage |
| | Intensity | Intensity | Intensity | Intensity | Intensity | Intensity | In the Box |
| S1 | 6 | 9.04 | 150.70 | 64 | 7.9 | 61.95 | 27.8 |
| S2 | 12 | 12.03 | 100.27 | 64 | 7.3 | 53.37 | 1.4 |
| S3 | 7 | 7.81 | 111.56 | 44 | 10.4 | 108.34 | 24.4 |
| S4 | 10 | 3.51 | 35.1183 | 11 | 15.0 | 226.09 | 96.3 |

Standard microbiological tests were used to confirm the presence of Listeria in S4 and the presence of non-Listeria in S1, S2 and S3. For samples S1, S2, S3, Gram stain and detection of the enzymes catalase and coagulase were performed. All three samples were Gram-positive cocci, catalase positive and coagulase negative. This confirmed that they were not Listeria species. Tests performed on S4 samples showed that bacteria in S4 were Gram-positive rods, catalase positive and motile (typical tumbling motility) at 22° C. but not at 37° C., confirming the identification of Listeria species. See Bergey's Manual of Determinative Bacteriology (9^{th} Ed., 1994, Williams & Wilkins Publishers).

Selection media, such as Bacto Fraser Broth (Difco, Becton Dickinson, Sparks, Md. 21152), may be used to enrich or select the growth of Listeria species.

### EXAMPLE 2

### Identification of Microorganisms Using a Signature Library

Two microorganisms labeled A and B were grown in Columbia Broth (Difco, Becton Dickinson, Sparks, Md. 21152) for 6 hours at 37° C. with gentle rocking. Serial dilutions of the cultures were then made in filtered (0.2 micron filter) 10 mM phosphate buffer (PB), pH 7.3, to an approximate concentration of 10⁵-10⁶ organisms per ml and stained for 5 minutes with 200 μM Draq5 at 37° C. in the dark. Samples were analyzed by flow cytometry using RBD2100. Profiles of A and B were recorded and analyzed. TABLE 5 summarizes the profiles of A and B.

TABLE 5
Profiles of Samples A and B

| Sample | A | B |
|---|---|---|
| Counts Percentage in Box 1 | 98% | 97.85% |
| Mean of Fluorescence Signals | 13.5 | 7.5 |
| in Box 1 | | |
| SD of Fluorescence Signals | 4.8 | 3.59 |
| in Box 1 | | |
| CV of Fluorescence Signals | 36.71 | 43 |
| in Box 1 | | |
| Mean of Scatter Signals | 14.5* | 11 |
| in Box 1 | | |
| SD of Scatter Signals | 8.95 | 13.62 |
| in Box 1 | | |
| CV of Scatter Signals | 80.12 | 124 |
| in Box 1 | | |

*this value does not match the corresponding value in the signature profile of TABLE 1.

Comparing to a signature library which comprises the signature profiles of TABLEs 1-3 and the signature profile used in FIG. 4, Sample A matched Enterococcus faecium except for the mean of scatter signals, while Sample B matched Listeria innocua. The identity of A and B thus determined was confirmed independently. This Example demonstrates that the applicability of this invention to microbial identification, and that expansion of the signature library to include various species of interest presents an opportunity for fast, easy microbial identification.

What is claimed:

1. A method for evaluating a microorganism, comprising:
(1) providing a plurality of said microorganism to a flow cytometer, the plurality of said microorganism being labeled with a fluorescent marker;
(2) obtaining from the flow cytometer fluorescence and scatter signals to generate a profile of said microorganism; and
(3) comparing the generated profile to a signature profile of a reference microorganism.

2. The method according to claim 1, wherein the generated profile of said microorganism comprises a bitmap of said microorganism, and the signature profile of said reference microorganism comprises a bitmap of said reference microorganism.

3. The method according to claim 2, wherein the generated profile is compared to the signature profile using a pattern recognition program.

4. The method according to claim 1, wherein the generated profile of said microorganism comprises a ratio of counts in a predetermined box in a bitmap of said microorganism over total counts in the bitmap of said microorganism, and wherein the signature profile of said reference microorganism comprises a range which encompasses a ratio of counts in the predetermined box in a bitmap of said reference microorganism over total counts in the bitmap of said reference microorganism.

5. The method according to claim 4, wherein the predetermined box is generated such that the ratio of counts in the predetermined box in the bitmap of said reference microorganism over total counts in the bitmap of said reference microorganism is about 90%.

6. The method according to claim 4, wherein the generated profile further comprises a mean, a standard deviation and a coefficient of variance of the fluorescence intensity in the predetermined box in the bitmap of said microorganism, and a mean, a standard deviation and a coefficient of variance of the scatter intensity in the predetermined box in the bitmap of said microorganism, and wherein the signature profile further comprises a first, second and third range which encompass a mean, a standard deviation and a coefficient of variance, respectively, of the fluorescence intensity in the predetermined box in the bitmap of said reference microorganism, and a fourth, fifth and sixth range which encompass a mean, a standard deviation and a coefficient of variance, respectively, of the scatter intensity in the predetermined box in the bitmap of said reference microorganism.

7. The method according to claim 4, wherein the generated profile is compared to the signature profile using a programmable processor.

8. The method according to claim 1, wherein said reference microorganism is selected from Listeria innocua, Enterococcus faecium, Pseudomonas aeruginosa and Candida albicans.

9. The method according to claim 1, wherein the fluorescent marker is not specific to said microorganism and said reference microorganism.

10. The method according to claim 9, wherein said microorganism is capable of causing food poisoning or food spoilage.

11. The method according to claim 1, wherein said microorganism is capable of causing a human, animal or plant disease.

12. The method according to claim 1, wherein said microorganism is a pharmaceutical product or a pharmaceutical contaminant.

13. The method according to claim 1, wherein said microorganism is obtained by culturing a sample taken from an environment of interest.

14. The method according to claim 1, comprising obtaining a plurality of said microorganism in a selection culture media.

15. The method according to claim 1, comprising comparing the generated profile to a plurality of signature profiles of reference microorganisms.

16. A method for evaluating a microorganism, comprising:
(1) providing a plurality of said microorganism to a flow cytometer, the plurality of said microorganism being labeled with a fluorescent marker;
(2) obtaining from a detecting means fluorescence and scatter signals to generate a profile of said microorganism; and
(3) comparing the generated profile to a signature profile of a reference microorganism using a comparing means.

17. The method according to claim 16, wherein said detecting means comprises a flow cytometer, and said comparing means comprises a programmable processor.

18. The method according to claim 16, wherein said comparing means comprises a means for pattern recognition.

19. The method according to claim 16, wherein said comparing means comprises a means for comparing a ratio to a range, wherein said ratio is a ratio of counts in a predetermined box in a bitmap of said microorganism over total counts in the bitmap of said microorganism, and wherein said range encompasses a ratio of counts in the predetermined box in a bitmap of said reference microorganism over total counts in the bitmap of said reference microorganism.

20. A system for evaluating a microorganism, comprising:
(1) a means for gathering fluorescence and scattering signals of said microorganism; and
(2) a means for comparing said fluorescence and scatter signals to a signature profile of a reference microorganism.

21. The system according to claim 20, wherein said means for gathering comprises a flow cytometer, and said means for comparing comprises a programmable processor.
